(19) United States
(12) Patent Application Publication
(10) Pub. No.: US 2013/0245485 A1
(43) Pub. Date: Sep. 19, 2013

(54) SYSTEM AND METHOD TO ASSESS CAUSAL SIGNALING IN THE BRAIN DURING STATES OF CONSCIOUSNESS

(71) Applicant: THE REGENTS OF THE UNIVERSITY OF MICHIGAN, Ann Arbor, MI (US)

(72) Inventors: George A. Mashour, Ann Arbor, MI (US); UnCheol Lee, Ann Arbor, MI (US)

(73) Assignee: THE REGENTS OF THE UNIVERSITY OF MICHIGAN, Ann Arbor, MI (US)

(21) Appl. No.: 13/804,706

(22) Filed: Mar. 14, 2013

Related U.S. Application Data
(60) Provisional application No. 61/612,514, filed on Mar. 19, 2012.

(51) Int. Cl. A61B 5/04; A61B 5/00; A61B 5/0476

(52) U.S. Cl. CPC A61B 5/04012; A61B 5/0476; A61B 5/4821; A61B 5/74
USPC 600/544

(57) ABSTRACT

A system and method for assessing causal signaling in the brain during states of consciousness are described. More specifically, a system and method for determining a directed functional connectivity in the brain wherein neurophysiologic correlates are analyzed with respect to feedback and/or feedforward activities to determine a directional feedback connectivity and/or a directional feedforward connectivity associated with a level of consciousness in the brain.

### CROSS REFERENCE TO RELATED APPLICATIONS

The present application claims the benefit of U.S. Provisional Application No. 61/612,514, filed on Mar. 19, 2012, which is hereby incorporated by reference herein in its entirety.

### TECHNICAL FIELD

This application is generally related to assessing brain activity and, more specifically, to a system and method for determining directional connectivity in a frontoparietal network.

### BACKGROUND

Visual processing within the brain follows a posterior-to-anterior path, i.e., feedforward, from the primary visual cortex to the temporal lobe (ventral stream) and frontal lobe (dorsal stream). This activity in the primary visual cortex and the subsequent feedforward processing is thought to mediate sensory processing, which may occur outside of consciousness. In addition, an anterior-to-posterior flow of information, i.e., feedback or recurrent processing, from the frontal cortex to other cortical regions is thought to mediate conscious experience. In other words, feedback processing, or a feedback pathway, is thought to be necessary for consciousness. As such, feedback processing has been discussed as a possible neural correlate of consciousness beyond the visual system.

Consistent with this possibility, preliminary evidence suggests that anesthetic-induced unconsciousness is associated with a selective inhibition of anterior-to-posterior, i.e., feedback, activity. Although studies using neuroimaging, high-density electroencephalography (EEG), and transcranial magnetic stimulation have significantly contributed to the understanding of how general anesthetics might suppress consciousness, such techniques are impractical for the routine intraoperative assessment of anesthetic depth in the millions of patients receiving general anesthetics each year. On the other hand, while some “awareness monitors” may be practical for routine use, they employ empirically-derived algorithms that are not grounded in the cognitive neuroscience of consciousness or general anesthesia.

Identifying a neural correlate or cause of consciousness (as well as other related states, e.g., sleep disorders, vegetative state) that can be routinely measured in surgical patients would be an important advancement in the field of mechanistic study and general anesthesia, which may further the development of more sophisticated brain monitors for patients.

### SUMMARY OF THE INVENTION

A system and method for assessing causal signaling in the brain during states of consciousness are disclosed herein. An example method includes monitoring feedback activity between a first region of the brain and a second region of the brain and analyzing the monitored feedback activity between the first region and the second region to calculate or determine a directional feedback connectivity. This directional feedback connectivity, which may be indicated to a user, may be indicative of an effective connectivity reflecting causal interactions between the first region and second region of the brain. A level of consciousness in the brain may be determined by a comparison of the determined directional feedback connectivity to a baseline consciousness level.

In another example embodiment, the method may also include monitoring a feedforward activity between the second region of the brain and the first region of the brain and analyzing the monitored feedforward activity to calculate or determine a directional feedforward connectivity. This directional feedforward connectivity may be indicative of an effective connectivity reflecting causal interactions between the second region and the first region of the brain. A level of consciousness in the brain may be determined by a comparison, or ratio, of the determined directional feedback connectivity and the determined directional feedforward connectivity.

In a further example embodiment, a system for determining the causal relationship of two regions of the brain includes an integrated monitoring system including a processor, a display device, and one or more sensors, wherein the one or more sensors are operatively coupled to the brain to monitor a feedback activity between a first region of the brain and a second region of the brain. More specifically, the one or more sensors are connected or attached to an individual's scalp and are capable of sensing or detecting brain activity, such as causal signaling. The system includes a memory coupled to the integrated monitoring system, and an analyzing routine stored on the memory, which when executed on the processor, analyzes the monitored feedback activity to calculate or determine a directional feedback connectivity. The system may include an indicating routine stored on the memory, which when executed on the processor, indicates a level of consciousness in the brain at an indicator, wherein the level of consciousness is at least partially dependent on the determined directional feedback connectivity.

In another example embodiment, the system may also include the one or more sensors operatively coupled to the brain to monitor the feedforward activity between the second region of the brain and the first region of the brain wherein the analyzing routine also analyzes the monitored feedforward activity to calculate or determine a directional feedforward connectivity. The indicating routine may indicate the level of consciousness in the brain through a comparison or ratio of the determined directional feedback connectivity and the determined directional feedforward connectivity.

If desired, monitoring the feedback and feedforward activity may include employing electroencephalography, and analyzing the feedback and feedforward activities to calculate or determine directional connectivity may include employing an analytic method or causal analysis, such as evolutional map approach, symbolic transfer entropy, normalized symbolic transfer entropy, directed phase lag index, Granger causality, etc. Additionally, indicating the directional connectivity between the first and second regions of the brain may include indicating a level of consciousness to a user. Also, analyzing a feedforward activity between the first region and the second region may include analyzing feedforward activity between a parietal region of the brain and a frontal region of the brain, or between the temporal region of the brain and a frontal region of the brain; and, analyzing a feedback activity between the second region and the first region may include analyzing feedback activity between the frontal region of the brain and the parietal region of the brain, or between the frontal region of the brain and the temporal region of the brain.

### BRIEF DESCRIPTION OF THE DRAWINGS

FIGS. 1A-1C illustrate a feedback and feedforward connectivity in a frontoparietal (frontal-parietal region) network calculated or determined by the evolutional map approach (EMA), where FIG. 1A depicts the asymmetry between feedback connectivity and feedforward connectivity in three states (i.e., baseline consciousness, induction, and anesthetized) using the EMA analysis method; and FIGS. 1B and 1C depict absolute values of feedback connectivity (FIG. 1B) and feedforward connectivity (FIG. 1C), respectively, across the three states, where the feedback dominance in the baseline was reduced due to inhibition of feedback phase modulation after induction. (The error bar denotes the standard error (*: p<0.05, **: p<0.01, n=18 patients).)

FIGS. 2A-2C illustrate a feedback and feedforward connectivity in a frontoparietal network calculated or determined by the symbolic transfer entropy (STE) approach, where FIG. 2A depicts the asymmetry between feedback connectivity and feedforward connectivity in three states (i.e., baseline consciousness, induction, and anesthetized) using the STE analysis method; and FIGS. 2B and 2C depict absolute values of feedback connectivity (FIG. 2B) and feedforward connectivity (FIG. 2C), respectively, across the three states, where the feedback dominance in the baseline was reduced due to inhibition of feedback STE after induction; however, feedforward STE values were also reduced in the anesthetized state. (The error bar denotes the standard error (*: p<0.05, ***: p<0.001, n=18 patients).)

FIGS. 3A-3C illustrate an analysis of asymmetry, feedback, and feedforward symbolic transfer entropy (STE) for propofol, where FIG. 3A depicts the asymmetry between feedback STE (FIG. 3B) and feedforward STE (FIG. 3C) for the propofol group (n=9 patients). (The error bar denotes the standard error (*: p<0.05).)

FIGS. 4A-4C illustrate an analysis of asymmetry, feedback, and feedforward symbolic transfer entropy (STE) for sevoflurane, where FIG. 4A depicts the asymmetry between feedback STE (FIG. 4B) and feedforward STE (FIG. 4C) for the sevoflurane group (n=9 patients). (The error bar denotes the standard error (*: p<0.05).)

FIG. 5 is a chart illustrating post-anesthetic recovery of feedback symbolic transfer entropy (STE); the schematic diagrams in the top row represent the changing asymmetry between feedback and feedforward STE over the five states—baseline consciousness, induction, anesthetized, recovery, and post recovery; a significant change in feedforward STE occurred only between anesthetized and post-recovery states (which is not presented in this figure). The feedback and feedforward STE are denoted with a striped and solid pattern, respectively, for each state. (The error bar denotes the standard error (*: p<0.05, **: p<0.01, ***: p<0.001, n=18 patients).)

FIGS. 6A-6F illustrate an analysis of feedforward connectivity, feedback connectivity, and asymmetry for three heterogeneous anesthetics using normalized symbolic transfer entropy (NSTE). The states of baseline consciousness (B) and anesthesia (A) are divided among three equal substates (B1, B2, B3 and A1, A2, A3). The feedback (▴) and feedforward (▪) connectivities (6A, 6B, and 6C) and their corresponding symmetry (6D, 6E, and 6F) in the frontal-parietal network are shown for ketamine (FIGS. 6A and 6D), propofol (FIGS. 6B and 6E), and sevoflurane (FIGS. 6C and 6F).

FIGS. 7A and 7B are graphs of power spectra and correlations of the frontal and parietal regions for the original and surrogate EEG, where the original (FIG. 7A) and surrogate (FIG. 7B) EEG data have the same power spectral densities for the frontal (solid lines) and parietal (dotted lines) regions for three states (baseline consciousness, induction, anesthetized). The distribution of linear correlation coefficients (the zeroth lag of the normalized covariance function) between frontal and parietal EEG channels has a positive mean value (inset of (FIG. 7A)), whereas the distribution for surrogate data has a zero mean value (inset of (FIG. 7B)), n=18 patients.

FIGS. 8A and 8B illustrate an estimation of bias caused by power spectral differences between frontal and parietal regions, where the biases caused by the power spectral difference between frontal and parietal regions were denoted with mean and standard error over 18 patients; for EMA (FIG. 8A), and for STE (FIG. 8B). Connectivity measures based on the original EEG data (feedback—squares (□), feedforward—circles (∘)) show that the biases do not account for changes across states; n=18 patients.

FIG. 9 is a flowchart of an exemplary method for assessing an individual's brain activity in accordance with the described embodiments.

FIG. 10 is a flowchart of another exemplary method for assessing an individual's brain activity in accordance with the described embodiments.

FIG. 11 illustrates an exemplary block diagram of a network and computer hardware that may be utilized in an exemplary system in accordance with the described embodiments.

FIG. 12 illustrates an exemplary block diagram of a computer system on which an exemplary system may operate in accordance with the described embodiments.

### DETAILED DESCRIPTION

The disclosed system and method utilize electroencephalography (EEG) in conjunction with analytical methods to analyze causal interaction, i.e., effective connectivity, between different regions of the brain. In particular, the analysis of feedforward and feedback connectivity between, for example, the frontal and parietal regions of the brain (e.g., a frontoparietal network) may provide a neurophysiologic correlate for anesthetic-induced unconsciousness, sleep disorders, vegetative state, etc.

It was determined through the study of the directionality of frontoparietal connectivity in human volunteers during consciousness, anesthesia (e.g., propofol), and recovery, that feedback connectivity in humans was dominant in the conscious state with respect to the feedforward connectivity. After induction with propofol however, both the feedforward and the feedback connectivities precipitously decreased, although the feedforward connectivity recovered to baseline consciousness during general anesthesia while the feedback connectivity remained suppressed until the return of consciousness.

To investigate the causal relationships between frontal and parietal regions of the brain, EEG data occurring through several states of consciousness were gathered and analyzed. EEG data were recorded at eight monopolar channels in the frontoparietal region ((Fp1, Fp2, F3, F4, T3, T4, P3, and P4 referenced by A2, which followed the international 10-20 system for electrode placement) by a WEEG-32 (LXE3232-RF, Laxtha Inc., Daejeon, Korea)) with a sampling frequency of 256 Hz. Electromyogram (EMG) was concurrently recorded at four bipolar channels ((bilateral frontalis and temporalis muscle) by a QEMG-4 (Laxtha Inc., Daejeon, Korea)) with a sampling frequency of 1024 Hz. The attached position of the four muscle electrode pairs followed the disclosure of Goncharova et al. (See Goncharova I I, McFarland D J, Vaughan T M, Wolpaw J R (2003) EMG contamination of EEG: Spectral and topographical characteristics. ClinNeurophysiol 114: 1580-1593.)

Recordings of the EEG and EMG were divided into five monitoring epochs: (i) baseline—before anesthetic induction and five minutes of recording; (ii) induction—from the start of anesthetic induction to the loss of consciousness; (iii) anesthetized state—from the loss of consciousness to five minutes after the loss of consciousness; (iv) recovery—from the end of anesthesia to the recovery of consciousness; and, (v) post-recovery—from admission to the recovery in the Post-Anesthesia Care Unit until five minutes after admission. The time to loss of consciousness and recovery of consciousness was determined by checking at five second intervals for failure to respond to a verbal command, e.g., open your eyes; and the recovery of individuals was defined by an Observer's Assessment of Alertness/Sedation Scale value being greater than five. One-minute periods of artifact-free EEG epochs were selected by visual inspection among five-minute durations of EEG epochs during the five states. EEG epochs coinciding with an increase of EMG amplitude and containing non-stationary wave changes in one-minute EEG epochs were excluded and Fourier-based band-pass filtering (0.5-55 Hz) was applied to the EEG data before the calculation of directionality.

Feedforward and feedback connectivities in the frontoparietal region were quantified based on digitized EEG data and analyzed to identify, determine, and/or assess a causal relationship. The causal relationship between two signals of the EEG reflects a directed functional connection in the brain. In other words, if the frontal activity was the cause of parietal activity, it was deemed a “feedback” connection; conversely, if the parietal activity was the cause of frontal activity, it was deemed a “feedforward” connection.

To assess the directional flow of information in the frontoparietal network during consciousness and anesthesia, several analytical methods based on different theoretical backgrounds may be employed: (i) evolutional map approach (EMA), which is based on the phase dynamics of two signals; (ii) transfer entropy (TE), which is based on information theory, particularly symbolic transfer entropy (STE) and normalized STE (NSTE).

For EMA, if it is assumed two EEG signals x_{1,2}(t) influence each other through weak coupling, then the weak coupling would be primarily manifested as an effect on the phases of EEG, rather than the amplitudes. EMA therefore measures the cross-dependence of coupled nonlinear oscillators based on their phase dynamics. The phases φ_{1,2} of signals x_{1,2}(t) were obtained by Hilbert transformation, and the phase increments Δ_{1,2}=φ_{1,2} (t+τ)−φ_{1,2} (t) may be calculated during time increment τ. The influence of x₂(t) on x₁(t) is estimated by the dependency of φ₂ on Δ₁. In practice, the phase increment may be expressed as a function of phases φ₁ and φ₂ by finite Fourier series:

$$\begin{matrix}{F}_{1} = {\sum }_{m , l} {A}_{m , l} {e}^{i m {φ}_{1} + i l {φ}_{2}} & ( 1 ) \\ {F}_{2} = {\sum }_{{m}^{′} , {l}^{′}} {A}_{{m}^{′} , {l}^{′}} {e}^{i {m}^{′} {φ}_{1} + i {l}^{′} {φ}_{2}} & ( 2 )\end{matrix}$$

where A_{m,l,m′,l′} were the coefficients and m, m′,l,l′=3 are set as optimal for the EEG.

The cross dependence between x₁ and x₂ are calculated as follows:

$${c}_{1 , 2}^{2} = \int {\int }_{0}^{2 π} {( \frac{\partial {F}_{1 , 2} ( {φ}_{1} , {φ}_{2} )}{\partial {φ}_{2 , 1}} )}^{2} d {φ}_{1} d {φ}_{2} \qquad ( 3 )$$

Here, c₁ is the influence of φ₂ to F₁ and c₂ is vice-versa. τ may be set as 1 s, considering that the time required for conscious processing is thought to exceed 270 ms. In order to avoid edge effects, the Hanning window (cosine half-wave) may be applied to the beginning and the end of one-minute-long EEG data (1.5 s on each end). After applying the Hilbert transform, the phase values of 1.5 s may be discarded on each side of the data. The reliability of the cross-dependence c_{i→j} and c_{j→i}, may be tested with models and application to empirical data. The directed functional connectivity, c_{f→p}, between two scalp areas may be defined as average cross dependences from one to the other scalp areas in both directions, and the mean directionality index d is a normalized form of the cross-dependences, which indicates the asymmetry of modulation:

$$\begin{matrix}{\overline{c}}_{f \to p} = \frac{1}{{m}_{f} {m}_{p}} {\sum }_{( i , j ) = 1}^{{m}_{f} {m}_{p}} {c}_{i \to j} & ( 4 ) \\ \overline{d} = \frac{1}{{m}_{f} {m}_{p}} {\sum }_{( i , j ) = 1}^{{m}_{f} {m}_{p}} {d}_{i , j} & ( 5 )\end{matrix}$$

where m_{f}=4 and mₚ=2 are the number of EEG channels on both scalp areas, respectively, and the index d_{i,j}=(c_{i→j}−c_{j→i})/(c_{i→j}+c_{j→i}) varies from 1 in the case of unidirectional coupling (i→j) to −1 in the opposite case (j→i) with intermediate values −1<d_{i,j}<1 corresponding to bidirectional coupling.

With respect to transfer entropy (TE), it offers a nonlinear and model-free estimation of directed functional connectivity based on information theory, quantifying the degree of dependence of Y on X or vice-versa. TE can be defined as the amount of mutual information between the past of X (X^{P}) and the future of Y (Y^{F}), when the past of Y (Y^{P}) is already known. For example,

TE_{X→Y}=I(Y^{F};X^{P}|Y^{P})=H(Y^{F}|Y^{P})=H(Y^{F}|X^{P},Y^{P}) (6)

where H(Y^{F}|Y^{P}) is the entropy of the process Y^{F} conditional on its past.

The distributions of X^{P}, Y^{P}, and Y^{F} can be written explicitly as

$$\begin{matrix}{TE}_{X \to Y} = Σ P ( {Y}^{F} \vert {Y}^{P} , {X}^{P} ) {log}_{2} [ \frac{P ( {Y}^{F} , {Y}^{P} , {X}^{P} ) P ( {Y}^{P} )}{P ( {Y}^{P} , {X}^{P} ) P ( {Y}^{F} , {Y}^{P} )} ] & ( 7 ) \\ I [ {Y}^{F} ; {X}^{P} , {Y}^{P} ] = I ( {Y}^{F} ; {Y}^{P} ) + {TE}_{X \to Y} & ( 8 )\end{matrix}$$

Equation (8) shows that the TE represents the amount of information provided by the additional knowledge of the past of X in the model describing the information between the past and the future of Y.

One disadvantage of TE is the subjective decision for the bin size in the probability calculation in equation (7). To avoid this problem, symbolic transfer entropy (STE) can be used. In STE, each vector for Y^{F}, X^{P}, and Y^{P} in equation (7) is a symbolized vector point. For instance, a vector Yₜ consists of the ranks of its components Yₜ=[y₁, y₂, . . . , yₘ], where yⱼ=y_{t-m×(j-1)τ} is replaced with the rank in ascending order, yⱼε[1, 2, . . . , m] for j=1, 2, . . . , m. Here m is the embedding dimension and τ is the time delay. STE is defined in the same way as equation (7), but replacing the embedded vector points with the symbolized vector points.

As compared to original transfer entropy, STE is advantageous in that it avoids binning the measured values in the probability calculation and may be considered a computationally more efficient method for quantifying the dominating direction of information flow between time series from structurally identical and non-identical coupled systems.

EMA and STE have different theoretical backgrounds (phase dynamics and information theory, respectively), and each method has its own set of advantages and disadvantages in the detection of causal relationships. By applying both methods to the EEG data, an estimate of the feedback and feedforward connectivities in the frontoparietal network during general anesthesia can be obtained in a more comprehensive manner.

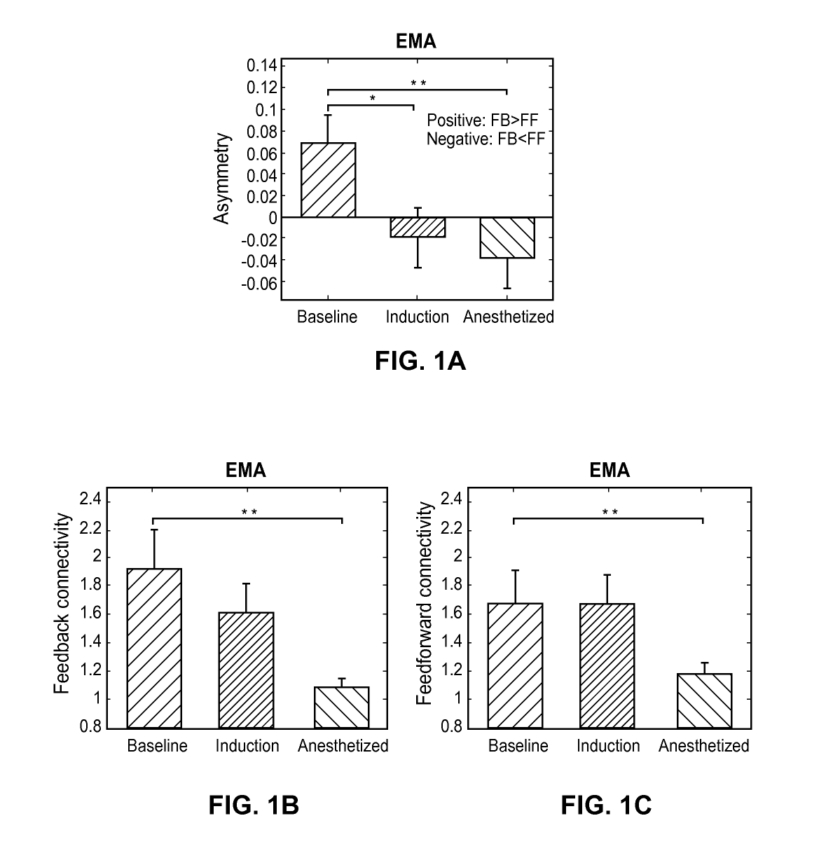

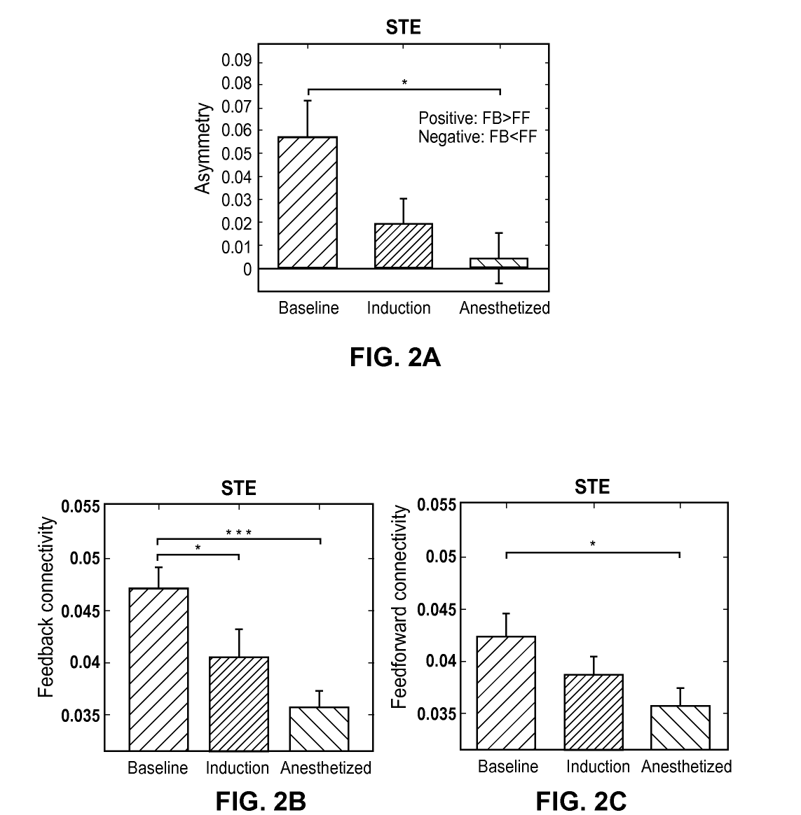

Referring now to the figures, FIGS. 1A-1C and 2A-2C depict the average feedback and feedforward connectivity and its asymmetry measured by the EMA (FIGS. 1A-1C) and STE (FIGS. 2A-2C) methods, respectively. Pairs of EEG channels between the two regions of the brain (Fp1, Fp2, F3, F4, and P3, P4) may be used for the calculation of bidirectional frontal-parietal connectivity. During baseline consciousness, the asymmetry between feedback connectivity and feedforward connectivity may be observed as shown in FIGS. 1A and 2A. By definition, the positive value of asymmetry in both measures indicates that feedback connectivity exceeds feedforward connectivity.

After induction of anesthesia, the asymmetry was significantly reduced as assessed by EMA, see FIG. 1A (p=0.0052, F=6.166, df=2 (states) and 17 (individuals), n=18; repeated measures one-way analysis of variance [ANOVA] with Tukey's multiple comparison test: p<0.05 for baseline and induction, p<0.01 for baseline and anesthetized). FIGS. 1B and 1C illustrate the individual means of feedback connectivity and feedforward connectivity, respectively, measured by the EMA method over three states—baseline consciousness, induction, and anesthetized. The feedback connectivity during baseline consciousness significantly decreased in the anesthetized state (p=0.0083, F=5.532, df=2 (states), 17 (individuals), n=18; repeated measures one-way ANOVA with Tukey's multiple comparison test: p<0.01 for baseline and anesthetized), while no significant difference in feedforward connectivity was observed.

The same procedure was applied to the EEG data using the STE method as was performed with the EMA method. The mean of asymmetry and the individual means of feedback and feedforward information flow are presented in FIGS. 2A-2C. The asymmetry of information flow between two brain regions is defined as STE_{f→p}−STE_{p→f} for each subject. Thus, positive values indicate the dominance of feedback connectivity, while negative values indicate the dominance of feedforward connectivity. Similar to EMA in the baseline, the STE feedback information flow was dominant with a significantly larger positive value in asymmetry as seen in FIG. 2A. This large asymmetric information flow was reduced in the anesthetized state (p=0.0295, F=3.914, df=2 (states), 17 (individuals), n=18; repeated measures one-way ANOVA with Tukey's multiple comparison test: p<0.05 for baseline and anesthetized), resulting in balanced information flows across two directions. The reduced asymmetry was caused by a reduction of feedback connectivity, even though there was also a significant reduction in feedforward flow, see FIGS. 2B and 2C (p=0.0001, F=11.72, df=2 (states) and 17 (individuals), n=18; repeated measures one-way ANOVA with Tukey's multiple comparison test: p<0.05 for baseline and induction, p<0.001 for baseline and anesthetized).

In contrast to the EMA method, the STE method detected significant suppression of feedback connectivity during anesthetic induction (p=0.0156, F=4.711, df=2 (states) and 17 (individuals), n=18; repeated measures one-way ANOVA with Tukey's multiple comparison test: p<0.05 for baseline and induction). However, despite the slightly different results in the analyzed states of consciousness and the associated feedback and feedforward connectivities in the frontoparietal network calculated by the EMA and STE, both EMA and STE demonstrate that preferential inhibition of feedback connectivity and reduction of feedback dominance during general anesthesia was consistent across both methods.

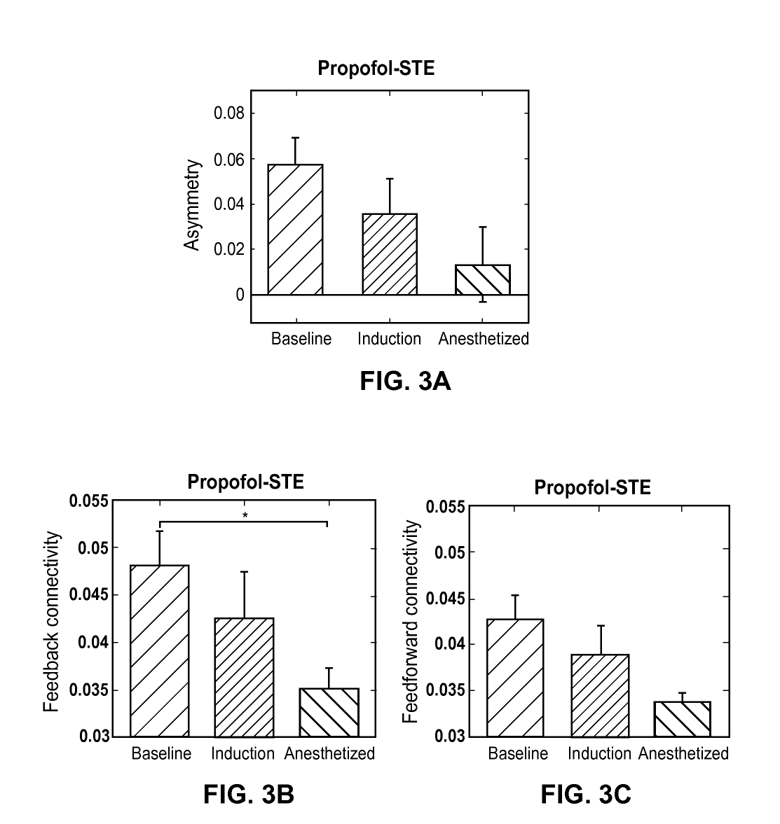

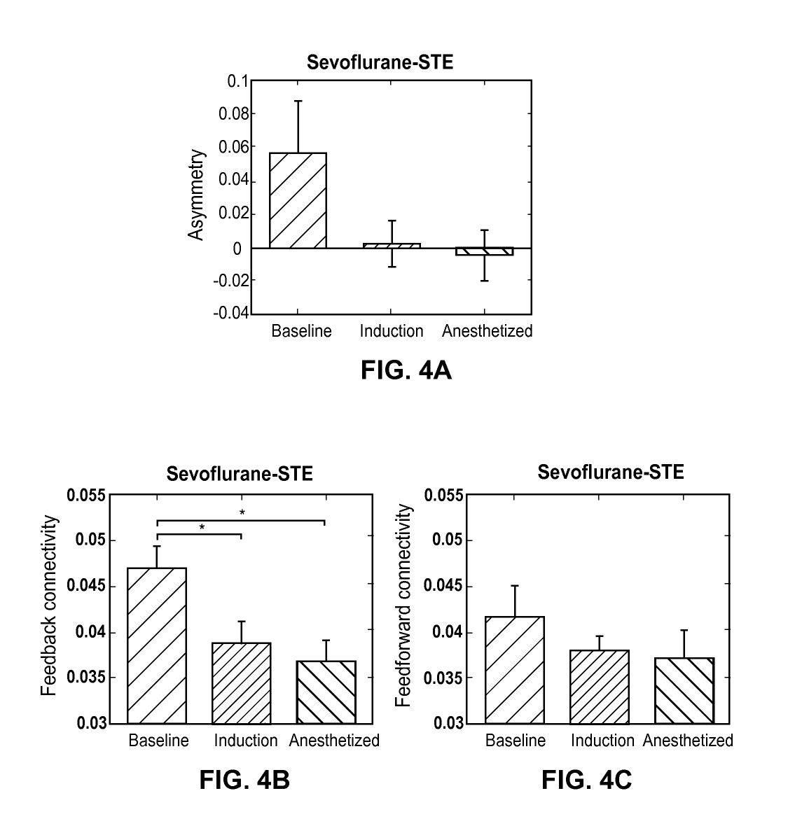

The effects of two anesthetics, i.e., propofol and sevoflurane, on feedback inhibition and the reduction of feedback/feedforward connectivity ratios were also analyzed individually using both the EMA and STE methods. Although the EMA method did not show any significant results primarily due to large individual variances over the three states, the trends were consistent with the STE method. The feedback and feedforward information flows measured by the STE method for the individual anesthetics demonstrated similar results to those of the combined data, see FIGS. 3A-3C (propofol) and 4A-4C (sevoflurane). In particular, the dominant feedback information flow during consciousness and the symmetrical flow during general anesthesia due to reduction of feedback connectivity were found for both anesthetics. (For feedback connectivity during propofol: p=0.0167, F=5.345, df=2 (states) and 8 (individuals), n=9; repeated measures one-way ANOVA with Tukey's multiple comparison test: p<0.05 for baseline and anesthetized; and for feedback connectivity during sevoflurane: p=0.004, F=7.946, df=2 (states) and 8 (individuals), n=9; repeated measures one-way ANOVA with Tukey's multiple comparison test: p<0.05 for baseline and induction, p<0.001 for baseline and anesthetized.) One observed difference between the two anesthetics was that sevoflurane produced a balanced information flow during anesthetic induction. This preceded the effect of propofol, which resulted in balanced information flow during the anesthetized state. This difference may be due to the fact that equisedative concentrations were not delivered during induction.

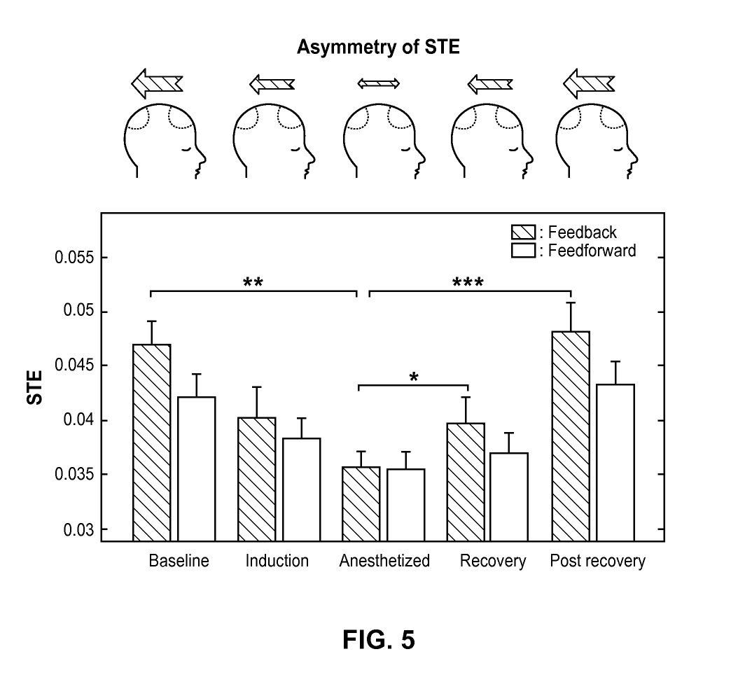

The return of dominant feedback connectivity measured by STE in the recovery and post-recovery state is illustrated in FIG. 5. The symmetric information flow during general anesthesia was disrupted during the recovery period (when anesthetic drug administration was terminated), but was not yet significant. The asymmetric feedback and feedforward information flows returned to the baseline level in the post-recovery state. (For feedback connectivity: p=0.0002, F=6.294, df=4 (states) and 17 (individuals), n=18; repeated measures one-way ANOVA with Tukey's multiple comparison test: p<0.01 for baseline and anesthetized, p<0.0001 for anesthetized and post-recovery, p<0.05 for recovery and post-recovery; and for feedforward connectivity: p=0.0059, F=3.976, df=4 (states) and 17 (individuals), n=18; repeated measures one-way ANOVA with Tukey's multiple comparison test: p<0.05 for anesthetized and post-recovery.)

To remove the bias of STE for a given EEG dataset, the shuffled data method may be implemented. The shuffled data retains the same signal characteristics as the original signal, but the causal relation is completely eliminated. This shuffling process may be applied only to the source signal (X), leaving the target signal (Y) intact. The STE with the shuffled source signal (X_{Shuff}^{P}), STE_{X→Y}^{Shuffled}=H(Y^{F}|Y^{P})−H(Y^{F}|X_{Shuff}^{P}, Y^{P}), estimates the bias caused by the signal characteristics of the source signal (X). The unbiased STE was normalized as:

$${NSTE}_{X \to Y} = \frac{{STE}_{X \to Y} - {STE}_{X \to Y}^{Shuffled}}{H ( {Y}^{F} \vert {Y}^{P} )} ∈ [ 0 , 1 ] \qquad ( 9 )$$

NSTE is normalized STE (dimensionless) in which the bias of STE is subtracted from the original STE and then divided by the entropy within the target signal, H(Y^{F}|Y^{P}). Intuitively, NSTE represents the fraction of information in the target signal Y not explained by its own past and explained by the past of the source signal X.

Additionally, the asymmetry between NSTE_{X→Y} and NSTE_{Y→X} was defined as:

$${DF}_{X \to Y} = \frac{{NSTE}_{X \to Y} - {NSTE}_{Y \to X}}{{NSTE}_{X \to Y} + {NSTE}_{Y \to X}} ∈ [ - 1 , 1 ] \qquad ( 10 )$$

Therefore, if DF_{X→Y} has a positive value, the connectivity from X to Y is dominant, and vice-versa for a negative value. The feedback and feedforward connections in the frontoparietal network were evaluated with NSTE_{f→p} and NSTE_{p→f} over the numerous subjects and heterogeneous anesthetics (i.e., 30 ketamine, 9 propofol, 9 sevoflurane). The average N̅S̅T̅E̅_{f→p} and N̅S̅T̅E̅_{p→f} were calculated over multiple pairs of EEG channels between the frontal and parietal regions for each subject;

$${\overline{NSTE}}_{f \to p} = \frac{1}{{n}_{f} \cdot {n}_{p}} {\sum }_{( i , j ) = 1}^{{n}_{f} , {n}_{p}} {NSTE}_{i \to j} ,$$

where n_{f}=4 and nₚ=2. The asymmetry of information flow between the two brain regions was defined as D̅F̅_{f→p} (equation (10)) for each subject.

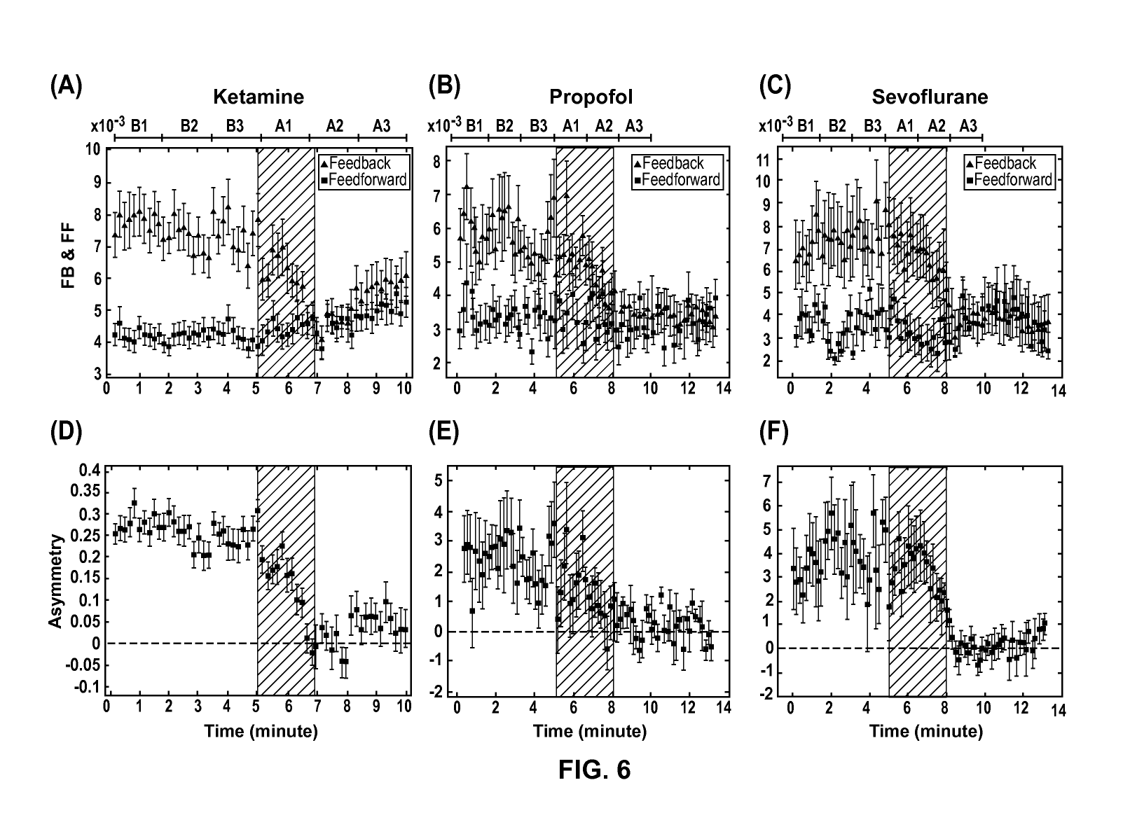

In FIGS. 6A-6F, the feedback and forward connections (FIGS. 6A-6C) and their respective asymmetry (FIGS. 6D-6F) in the fronto-parietal network are shown for ketamine (FIGS. 6A and 6D), propofol (FIGS. 6B and 6E) and sevoflurane (FIGS. 6C and 6F). The means and standard errors are denoted at each window and anesthetic administration is highlighted by the diagonally-lined vertical portion of each graph. Six substates (i.e., B1, B2, and B3 in baseline consciousness state, and A1, A2, and A3 in anesthesia) are depicted, wherein each substate includes ten 10 s EEG epochs.

During ketamine anesthesia, it was found that NSTE_{f→p} and NSTE_{p→f} have multiscale properties, showing distinct information transfer between frontal-parietal regions in short- and long-term scales. This may be associated with simultaneous increases of gamma and delta powers (relatively short- and long-term dynamics). Therefore, information transmission of a single time scale would not be able to represent the characteristic multiscale connectivity of ketamine anesthesia. It was also observed that the maximum information transfer between frontal and parietal regions provides a consistent connectivity feature among ketamine, propofol and sevoflurane. Three embedding parameters—embedding dimension (d_{E}), time delay (τ), and prediction time (δ)—are needed for NSTE. The parameter set that provides the maximum information transfer (NSTE) from the source signal to the target signal was selected as the primary connectivity for a given EEG dataset, instead of applying a conventional embedding method. By investigating the NSTE in the broad parameter space of d_{E} (from 2 to 10) and τ (from 1 to 30), the embedding dimension (d_{E}) was fixed at 3, which is the smallest dimension providing a similar NSTE, to find the time delay (τ) producing maximum NSTE. In this parameter space, a vector point could cover from 11.7 ms (with τ=1 and d_{E}=3) to 351 ms maximally (with τ=30 and d_{E}=3). If a parameter set for maximum information transfer was determined in one direction, the same parameters were used for the opposite direction. Taking the maximum NSTE as the primary connectivity for a given EEG dataset, all other processes are nonparametric without subjective decisions for embedding parameters. The prediction time was determined with the time lag (from 1 to 100, 3.9-390 ms) resulting in maximum cross-correlation, assuming the time lag as the interaction delay between the source and target signals.

The inhibition of asymmetry between the feedback and feedforward connectivity for ketamine, propofol, and sevoflurane can be seen in FIGS. 6A-6F and is a common feature found across these three heterogeneous anesthetics. Thus, it may be possible to determine a common neural correlate of anesthetic-induced unconsciousness for at least these three anesthetics.

A potential problem in estimating causal relationships is that spurious causality may result if two signals have significantly different spectral contents. Because of this concern for spurious feedback and feedforward connectivity derived from the difference of power spectra between the frontal and parietal brain regions, the potential spurious connectivity was estimated by using the surrogate data method. Surrogate data have precisely the same spectral contents as those of the original EEG data set, but their phases are randomly shuffled. Thus, true connections were removed by phase randomization between two EEG data sets; and any non-zero value resulting from connectivity analysis would therefore estimate a bias caused by power spectral differences.

To generate the surrogate data, the amplitude spectrum and amplitude distribution adjustment method was used. Twenty surrogate data sets were generated for each minute of EEG data. The average feedforward and feedback connections using EMA and STE were estimated with 160 pairs of surrogate data for several (e.g., 8) pairs of EEG channels between the frontal and parietal regions.

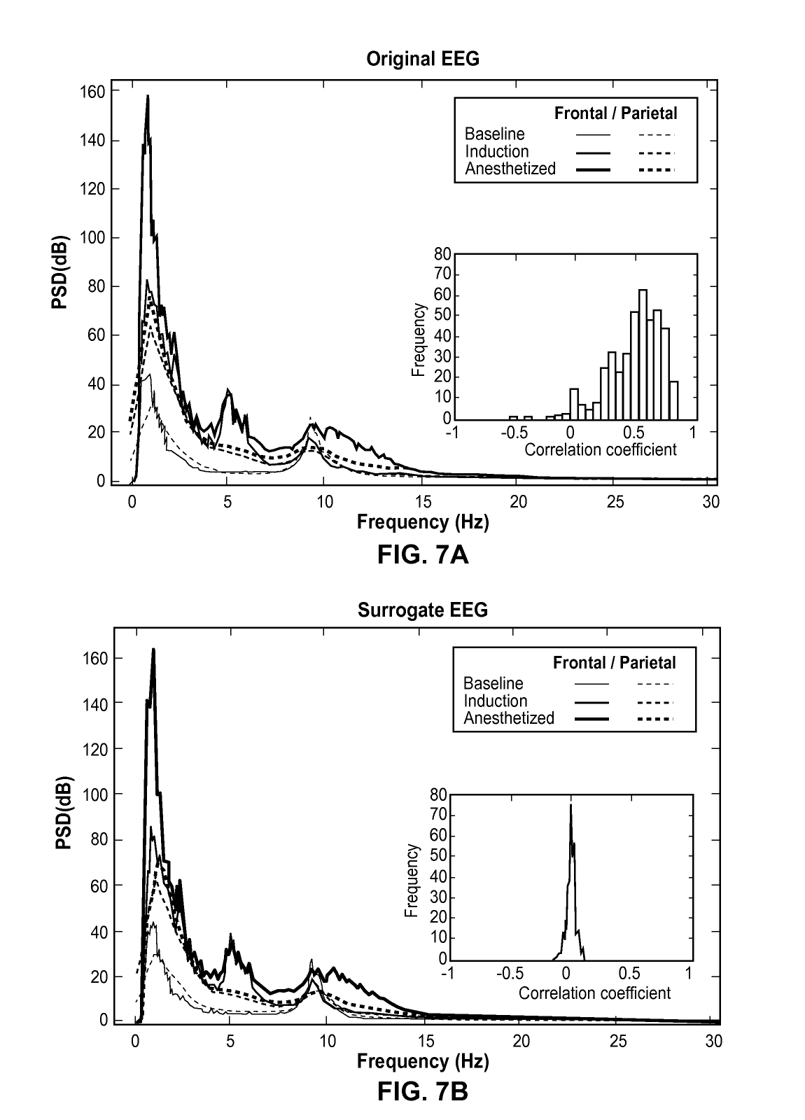

The average power spectral density was computed based on the Welch spectral estimator (MATLAB signal processing toolbox, “psd.m” with options: “spectrum.welch” with Hamming window and window size of 256). The average power spectral densities of EEG data for frontal (Fp1, Fp2, F3 and F4) and parietal (P3 and P4) regions across three states of consciousness in 18 patients are shown in FIG. 7A (solid lines for frontal, and dotted lines for parietal). The average power spectral densities of the corresponding surrogate data of the frontal and parietal EEG are demonstrated in FIG. 7B. The insets of FIGS. 7A and 7B demonstrate the histograms of linear correlation coefficients (the zeroth lag of the normalized covariance function) between frontal and parietal regions over 18 patients for the original EEG and surrogate data sets. The surrogate data set has the same power spectra as that of the frontal and parietal EEGs for the baseline consciousness, induction, and anesthetized states. The distribution of correlation coefficients of the original EEG data between frontal and parietal regions has a large positive mean (see inset of FIG. 7A). As expected, the distribution of correlation coefficients for the surrogate data has a mean of zero (see inset of FIG. 7B). Anesthetic induction generated increased power of lower frequency bands, particularly in the frontal region, which is a typical spectral change in the anesthetized state.

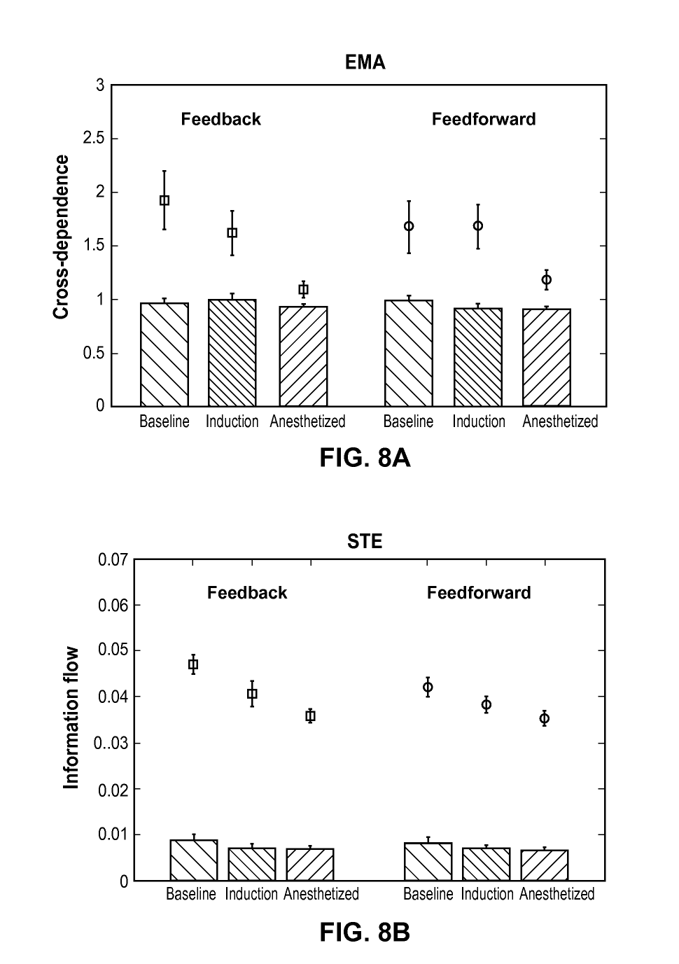

FIGS. 8A and 8B show the feedback and feedforward connections measured by EMA and STE using the surrogate data. The surrogate data of frontal and parietal EEG have non-zero EMA and STE values, which reflect estimates of spurious feedback and feedforward measures due to spectral changes. However, the bias based on the power spectra does not fully account for the EMA and STE values measured in the original data and furthermore does not change across states. As such, preferential inhibition of feedback connectivity and reduction of feedback/feedforward connectivity ratios is not solely attributable to changes in spectral contents.

Additional analysis methods may also be utilized to determine functional connectivity or directed connectivity to facilitate the determination of a consciousness level in the brain. For example, phase lag index (PLI) may be used to determine or estimate functional connectivity between EEG sensors. The PLI has been demonstrated to be robust with respect to the choice of references and less affected by volume conduction compared to other measures such as correlation and phase synchrony. The phase of EEG signals may be calculated by Hilbert transformation and the phase differences between EEG sensors i and j may be obtained for each time index (Δφₜ, t=1, 2, . . . , n). The PLI measure the asymmetry of the phase difference distribution by averaging the signs of phase differences.

PLIᵢⱼ=| [symbol] (sign(Δφₜ) [symbol] |,0≦PLIᵢⱼ≦1 (11)

For perfect phase locking, PLI is 1, if there is no consistent phase locking, PLI goes to 0. Thus, PLI ranges between 0 and 1. However, since this results in an absolute value, PLI loses information about phase lead and lag relationship between two signals.

Directed PLI (dPLI) is an analysis method that may capture directed connectivity by measuring the phase lag and lead relationship between two signals. Determination or calculation of the dPLI is almost the same as the calculation of the PLI. By applying Heaviside step function (where H(x)=1 if x>0, H(x)=0.5 if x=0, and H(x)=0 otherwise) to the phase difference and averaging it across all time steps, the dPLI of signal i with respect to j can be obtained.

dPLIᵢⱼ= [symbol] H(Δφₜ) [symbol] (12)

Regarding the phase lead and lag, as the signal i leads signal j, 0.5<dPLIᵢⱼ≦1, otherwise, if signal i is lagged by signal j, 0≦dPLIᵢⱼ<0.5. dPLI and PLI have the following relation:

PLIᵢⱼ=2|0.5−dPLIᵢⱼ| (13)

PLI may be used for undirected functional network analysis and dPLI may be used for directed functional connectivity. To remove a potential bias of dPLI from finite size effect (caused by lower frequency power spectra in anesthesia), the unbiased functional connection in the network may be defined with surrogate data, for example, 20 surrogate data sets generated from each subject's EEG recordings. The surrogate data set has the same power spectrum and histogram as that of the original EEG data, but with randomized phases after Fourier transformation. For a connection pair of i and j, if distribution of 20 dPLI values of surrogate data are deviated from dPLI of original data, the pair of i and j was deemed to be a true connection. Otherwise, the pair of i and j was considered to be disconnected (dPLIᵢⱼ=0.5). A nonparametric Wilcoxon signed rank test was performed so that the median of 20 dPLI values of surrogate data was compared to the dPLI of original data. (H₀(null-hypothesis): 20 dPLI values of surrogate data (dPLIᵢⱼ^{surrogate}) have symmetric distribution with median μ, where μ is the dPLI of original data (dPLIᵢⱼ^{original}).)

dPLIᵢⱼ=dPLIᵢⱼ^{original}−median(dPLIᵢⱼ^{surrogate})+0.5,if p<0.05 (14)

dPLIᵢⱼ=0.5,otherwise (15)

An undirected, weighted functional network may be obtained by transforming the dPLI matrix to the PLI matrix via Equation (13). In one embodiment, the densities of networks were 0.68+/−0.11 for wakefulness, 0.69+/−0.10 for loss of consciousness (LOC), and 0.68+\−0.09 for return of consciousness (ROC). The same network measures were tested for fully-connected weighted networks without generating surrogate data and there were no qualitative differences in the results between the two schemes. The PLI and dPLI analyses was conducted with MATLAB® (The MathWorks Inc., Natick, Mass.).

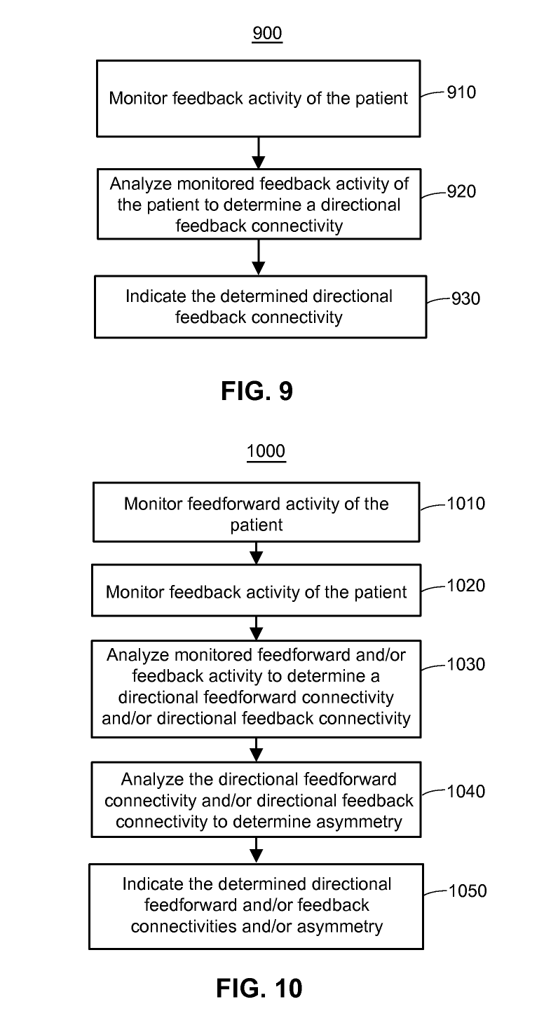

FIG. 9 illustrates a block diagram of an exemplary computer-implemented method 900 for assessing causal relationship in a frontoparietal network. The method 900 may include monitoring (block 910) feedback information (e.g., activity) associated with the patient. In one embodiment, the monitoring is performed through the use of electroencephalography (EEG) and results in EEG data. The method 900 analyzes the EEG data, e.g., via EMA, STE, NSTE (block 920) to determine a directional feedback connectivity. The method 900 may utilize the determined directional feedback connectivity to provide an output (block 930). The output may be a value or a signal to indicate the determined directional feedback connectivity, which may be associated with a level of consciousness in the brain. The value may be an absolute value or a comparison of the determined directional feedback connectivity with a baseline directional feedback connectivity, which may have been previously attained while the patient was in a conscious state. Importantly, this analysis of a frontoparietal network is not limited to levels of anesthetic-induced unconsciousness, but may also be applied to other regions of the brain as a system for dynamical analysis of sleep disorders, vegetative state, etc. Furthermore, the measured brain regions may extend beyond the frontoparietal areas to, for example, the frontotemporal network.

FIG. 10 illustrates a block diagram of another exemplary computer-implemented method 1000 for assessing causal relationship in a frontoparietal network. The method may include monitoring feedforward activity (block 1010) and feedback activity (block 1020) associated with the patient. The method 1000 may analyze the monitored feedforward and feedback activities, e.g., EEG data, to determine or calculate a directional feedforward connectivity and/or a directional feedback connectivity (block 1030). The method 1000 may analyze or utilize the directional feedforward connectivity and the directional feedback connectivity to determine or calculate asymmetry between the directional feedback connectivity and the directional feedforward connectivity (block 1040). The method 1000 may utilize EMA, STE, NSTE, PLI, and/or dPLI to analyze the feedforward and feedback activities to determine or calculate the feedforward and/or feedback directional connectivities and/or the asymmetry between the directional feedback and feedforward connectivities. The method 1000 may utilize the determined feedforward and feedback directional connectivities and/or the asymmetry between the directional feedback and feedforward connectivities to provide an indicator, e.g., output, indicating the determined feedforward and/or feedback directional connectivities and/or the asymmetry to a user (block 1050). The output may provide an indication relating to the level of consciousness in the brain of a patient and may be a value or signal. The value may be an absolute value or a comparison of the determined directional feedback connectivity (“FB”) to the determined directional feedforward connectivity (“FF”). The comparison, or ratio, may be expressed in any desired format to emphasize various aspects of the determined directional connectivities. Some example comparisons or ratios include, and are not limited to: a direct ratio of the determined directional feedback connectivities, (FB/FF); a percentage of excess determined directional feedback connectivity, (FB/FF−1)×100; and, a ratio of symmetry between the determined directional feedback and feedforward connectivities over the total amount of information transferred in both directions, (FB−FF)/(FB+FF).

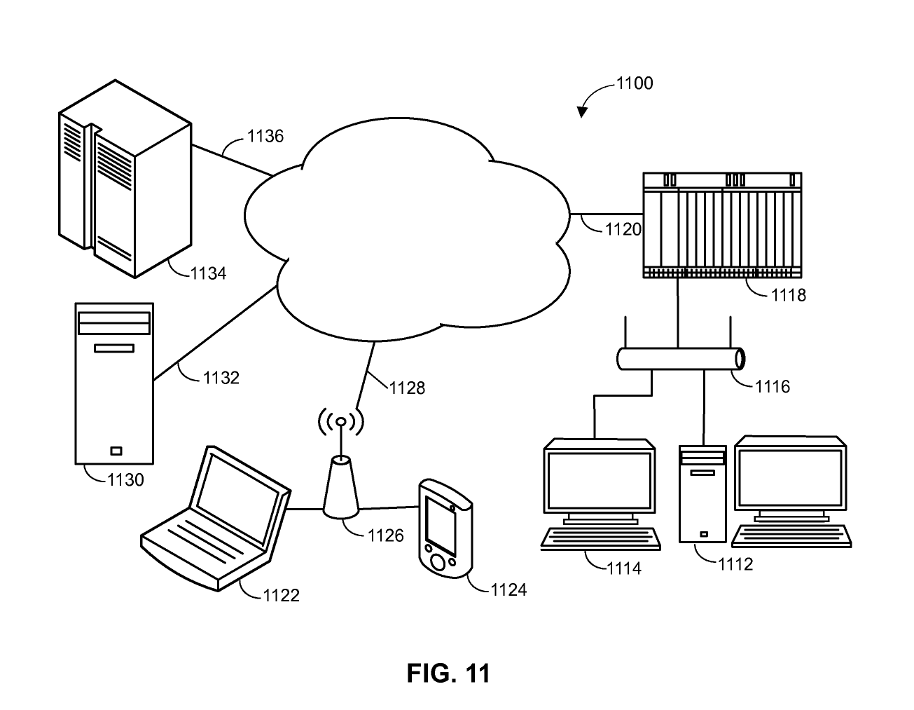

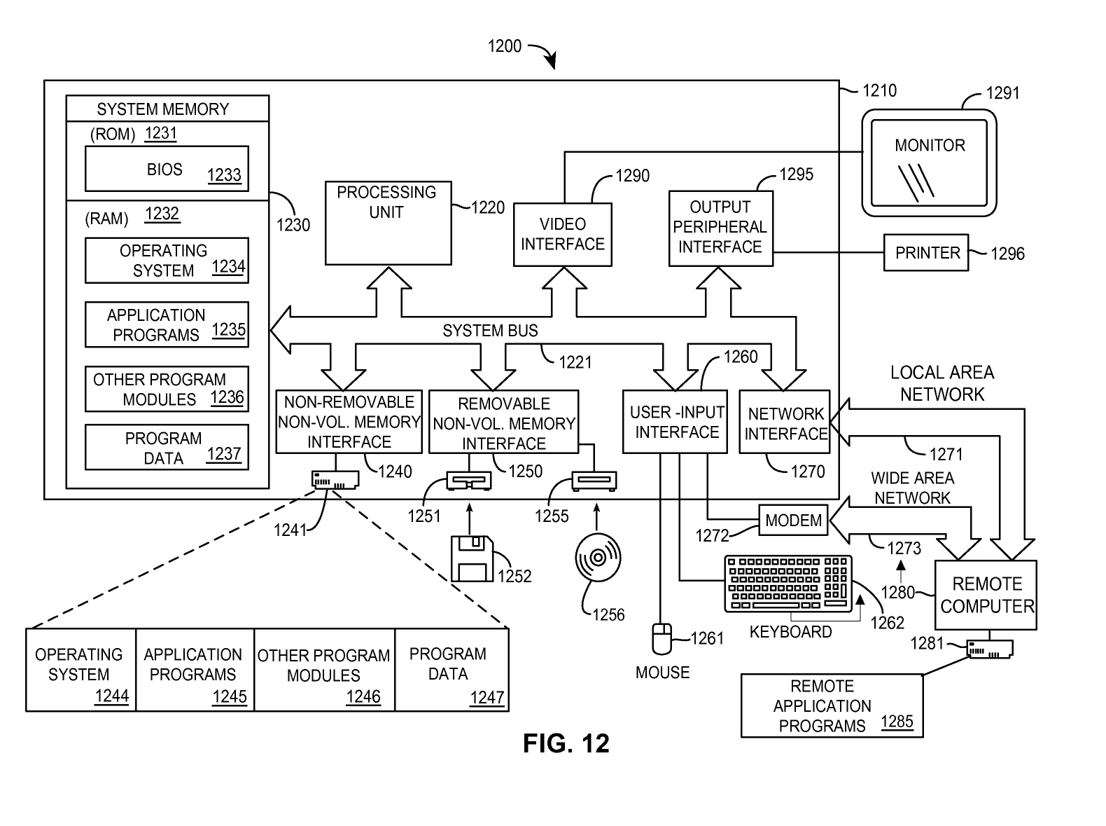

The assessment of effective connectivity in the brain may be generated using an electronic system. FIGS. 11 and 12 provide an exemplary structural basis for the network and computational platforms related to such a system.

FIG. 11 illustrates an exemplary block diagram of a network 1100 and computer hardware that may be utilized in an exemplary system for assessing causal signaling in the brain during states of consciousness in accordance with the described embodiments. The network 1100 may be the Internet, a virtual private network (VPN), or any other network that allows one or more computers, communication devices, databases, etc., to be communicatively connected to each other. The network 1100 may be connected to a personal computer 1112, and a computer terminal 1114 via an Ethernet 1116 and a router 1118, and a landline 1120. The Ethernet 1116 may be a subnet of a larger Internet Protocol network. Other networked resources, such as projectors or printers (not depicted), may also be supported via the Ethernet 1116 or another data network. Additionally, the network 1100 may be wirelessly connected to a laptop computer 1122 and a personal data assistant 1124 via a wireless communication station 1126 and a wireless link 1128. Similarly, a server 1130 may be connected to the network 1100 using a communication link 1132 and a mainframe 1134 may be connected to the network 1100 using another communication link 1136. The network 1100 may be useful for supporting peer-to-peer network traffic. The patient's monitored neurological information may also be received from a remotely-accessible, free-standing memory device (not shown) on the network 1100. In some embodiments, the patient's monitored neurological information may be received by more than one computer. In other embodiments, the patient's monitored neurological information may be received from more than one computer and/or remotely-accessible memory device.

Some or all calculations performed in the determination of a patient's effective connectivity described above (e.g., EMA, STE, NSTE, PLI, and/or dPLI analysis of feedback and feedforward activities to determine directional feedback and feedforward connectivities) may be performed by a computer such as the personal computer 1112, laptop computer 1122, server 1130 or mainframe 1134, for example. In some embodiments, some or all of the calculations may be performed by more than one computer.

Indicating a level of consciousness in the brain as described above in the embodiments may also be performed by a computer such as the personal computer 1112, laptop computer 1122, server 1130 or mainframe 1134, for example. The indications may be made by setting the value of a data field, for example. In some embodiments, indicating a level of consciousness may include sending data over a network such as network 1100 to another computing device.

FIG. 12 illustrates an exemplary block diagram of a system 1200 on which an exemplary method for assessing causal signaling in the brain during states of consciousness may operate in accordance with the described embodiments. The system 1200 of FIG. 12 includes a computing device in the form of a computer 1210. Components of the computer 1210 may include, and are not limited to, a processing unit 1220, a system memory 1230, and a system bus 1221 that couples various system components including the system memory to the processing unit 1220. The system bus 1221 may be any of several types of bus structures including a memory bus or memory controller, a peripheral bus, and a local bus using any of a variety of bus architectures. By way of example, and not limitation, such architectures include the Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus (also known as Mezzanine bus).

The computer 1210 typically includes a variety of computer readable media. Computer readable media can be any available media that can be accessed by computer 1210 and includes both volatile and nonvolatile media, and both removable and non-removable media. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, FLASH memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 1210. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared and other wireless media. Combinations of any of the above are also included within the scope of computer readable media.

The system memory 1230 includes computer storage media in the form of volatile and/or nonvolatile memory such as read only memory (ROM) 1231 and random access memory (RAM) 1232. A basic input/output system 1233 (BIOS), containing the basic routines that help to transfer information between elements within computer 1210, such as during start-up, is typically stored in ROM 1231. RAM 1232 typically contains data and/or program modules or routines, e.g., analyzing, calculating, indicating, etc., that are immediately accessible to and/or presently being operated on by processing unit 1220. By way of example, and not limitation, FIG. 12 illustrates operating system 1234, application programs 1235, other program modules 1236, and program data 1237.

The computer 1210 may also include other removable/non-removable, volatile/nonvolatile computer storage media. By way of example only, FIG. 11 illustrates a hard disk drive 1241 that reads from or writes to non-removable, nonvolatile magnetic media, a magnetic disk drive 1251 that reads from or writes to a removable, nonvolatile magnetic disk 1252, and an optical disk drive 1255 that reads from or writes to a removable, nonvolatile optical disk 1256 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 1241 is typically connected to the system bus 1221 through a non-removable memory interface such as interface 1240, and magnetic disk drive 1251 and optical disk drive 1255 are typically connected to the system bus 1221 by a removable memory interface, such as interface 1250.

The drives and their associated computer storage media discussed above and illustrated in FIG. 12 provide storage of computer readable instructions, data structures, program modules and other data for the computer 1210. In FIG. 12, for example, hard disk drive 1241 is illustrated as storing operating system 1244, application programs 1245, other program modules 1246, and program data 1247. Note that these components can either be the same as or different from operating system 1234, application programs 1235, other program modules 1236, and program data 1237. Operating system 1244, application programs 1245, other program modules 1246, and program data 1247 are given different numbers here to illustrate that, at a minimum, they are different copies. A user may enter commands and information into the computer 1210 through input devices such as a keyboard 1262 and cursor control device 1261, commonly referred to as a mouse, trackball or touch pad. A screen 1291 or other type of display device is also connected to the system bus 1221 via an interface, such as a graphics controller 1290. In addition to the screen 1291, computers may also include other peripheral output devices such as printer 1296, which may be connected through an output peripheral interface 1295.

The computer 1210 may operate in a networked environment using logical connections to one or more remote computers, such as a remote computer 1280. The remote computer 1280 may be an integrated monitoring system operatively coupled to an individual via an input/output component or device, e.g., one or more sensors capable of being connected or attached to the individual's scalp and detecting brain activity. The logical connections depicted in FIG. 12 include a local area network (LAN) 1271 and a wide area network (WAN) 1273, but may also include other networks. Such networking environments are commonplace in hospitals, offices, enterprise-wide computer networks, intranets, and the Internet.

When used in a LAN networking environment, the computer 1210 is connected to the LAN 1271 through a network interface or adapter 1270. When used in a WAN networking environment, the computer 1210 typically includes a modem 1272 or other means for establishing communications over the WAN 1273, such as the Internet. The modem 1272, which may be internal or external, may be connected to the system bus 1221 via the input interface 1260, or other appropriate mechanism. In a networked environment, program modules depicted relative to the computer 1210, or portions thereof, may be stored in the remote memory storage device 1281. By way of example, and not limitation, FIG. 11 illustrates remote application programs 1285 as residing on memory device 1281.

The communications connections 1270, 1272 allow the device to communicate with other devices. The communications connections 1270, 1272 are an example of communication media. The communication media typically embodies computer readable instructions, data structures, program modules or other data in a modulated data signal such as a carrier wave or other transport mechanism and includes any information delivery media. A “modulated data signal” may be a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Computer readable media may include both storage media and communication media.

The embodiments for the methods of assessing a causal relationship described above may be implemented in part or in their entirety using one or more computer systems such as the computer system 1200 illustrated in FIG. 12. The monitored neurological information, database, and/or models may be received by a computer such as the computer 1210, for example. The monitored neurological information, database, and/or models may be received over a communication medium such as local area network 1271 or wide area network 1273, via network interface 1270 or user-input interface 1260, for example. As another example, the monitored neurological information, database, and/or models may be received from a remote source such as the remote computer 1280 where the data is initially stored on memory device such as the memory storage device 1281. As another example, the monitored neurological information, database, and/or models may be received from a removable memory source such as the nonvolatile magnetic disk 1252 or the nonvolatile optical disk 1256. As another example, the monitored neurological information, database, and/or models may be received as a result of a human entering data through an input device such as the keyboard 1262.

Some or all analyzing or calculating performed in the determination of a patient's level of consciousness or a directed functional connectivity described above (e.g., analysis and calculations for determining directional feedforward connectivity and directional feedback connectivity) may be performed by a computer such as the computer 1210, and more specifically may be performed by one or more processors, such as the processing unit 1220, for example. In some embodiments, some calculations may be performed by a first computer such as the computer 1210 while other calculations may be performed by one or more other computers such as the remote computer 1280. The analyses and/or calculations may be performed according to instructions that are part of a program such as the application programs 1235, the application programs 1245 and/or the remote application programs 1285, for example.

Determining a patient's level of consciousness or directed functional connectivity as described above in the embodiments may also be performed by a computer such as the computer 1210. The indications may be made by setting the value of a data field stored in the ROM memory 1231 and/or the RAM memory 1232, for example. In some embodiments, indicating a patient's directional feedback and/or feedforward connectivity to a user may include sending data over a network such as the local area network 1271 or the wide area network 1273 to another computer, such as the remote computer 1281. In other embodiments, indicating a patient's feedback connectivity to a user may include sending data over a video interface such as the video interface 1290 to display information relating to the prediction on an output device such as the screen 1291 or the printer 1296, for example.

In conclusion, preferential inhibition of frontoparietal feedback connectivity and reduction of the feedback/feedforward connectivity ratio appears to be a clinically relevant neurophysiologic correlate of general anesthesia in surgical patients. The results described herein may be generalized to the perioperative setting because feedback connectivity inhibition was shown across several different classes of anesthetics, multiple analytic techniques, and a heterogeneous mix of patients. Additionally, analysis of frontoparietal feedback connectivity in relatively few EEG channels may be able to distinguish different phases of surgical anesthesia. Similar analysis of frontoparietal feedback connectivity may also be applicable to the assessment of sleep disorder, vegetative state, etc., where “feedforward” and/or “feedback” connectivity may appear between frontoparietal, or other regions, e.g., frontal and temporal lobes, of the brain.

We claim:

1. A method for assessing causal signaling in the brain during states of consciousness, the method comprising:
monitoring a feedback activity between a first region of the brain and a second region of the brain;
analyzing, via a processor in a computer system having a memory, the monitored feedback activity between the first region and the second region to determine a directional feedback connectivity; and,
indicating to a user, via the processor, a level of consciousness in the brain based on the directional feedback connectivity.

2. The method of claim 1, wherein monitoring a feedback activity includes employing electroencephalography (EEG) to attain EEG data.

3. The method of claim 2, wherein analyzing the monitored feedback activity includes employing an evolutional map approach analysis to analyze the EEG data.

4. The method of claim 2, wherein analyzing the monitored feedback activity includes employing a symbolic transfer entropy analysis or a normalized symbolic transfer entropy analysis to analyze the EEG data.

5. The method of claim 2, wherein analyzing the monitored feedback activity includes employing a directed phase lag index analysis to analyze the EEG data.

6. The method of claim 1, wherein indicating the level of consciousness in the brain includes comparing the directional feedback connectivity to a baseline directional feedback connectivity.

7. The method of claim 1, wherein analyzing the monitored feedback activity between the first region and the second region includes analyzing the monitored feedback activity between a frontal region of the brain and a parietal region of the brain.

8. The method of claim 1, further comprising:
monitoring a feedforward activity between the second region of the brain and the first region of the brain; and
analyzing, via the processor, the monitored feedforward activity to determine a directional feedforward connectivity, wherein the second region is a parietal region of the brain and the first region is a frontal region of the brain.

9. The method of claim 8, wherein indicating the level of consciousness in the brain includes comparing the directional feedback connectivity to the directional feedforward connectivity.

10. A system for assessing causal signaling in the brain during states of consciousness, the system comprising:
an integrated monitoring system including a processor, a display device, and one or more sensors, the one or more sensors operatively coupled to the brain to monitor a feedback activity between a first region of the brain and a second region of the brain;
a memory coupled to the integrated monitoring system;
an analyzing routine stored on the memory, which when executed on the processor, analyzes the monitored feedback activity to determine a directional feedback connectivity; and,
an indicating routine stored on the memory, which when executed on the processor, indicates a level of consciousness in the brain to a user at an indicator, wherein the level of consciousness in the brain is based on the directional feedback connectivity.

11. The system of claim 10 wherein the integrated monitoring system utilizes electroencephalography (EEG) to attain EEG data.

12. The system of claim 10, wherein the analyzing routine utilizes an evolution map approach analysis to analyze the monitored feedback activity.

13. The system of claim 10, wherein the analyzing routine utilizes a symbolic transfer entropy analysis or a normalized symbolic transfer entropy analysis to analyze the monitored feedback activity.

14. The method of claim 10, wherein the analyzing routine utilizes a directed phase lag index analysis to analyze the monitored feedback activity.

15. The system of claim 10, wherein at least one of the one or more sensors is operatively coupled to a frontal region of the brain and at least another of the one or more sensors is operatively coupled to a parietal region of the brain.

16. The system of claim 10, wherein the indicator indicates to a user a level of consciousness based on the directed functional connectivity.

17. The system of claim 10, wherein the level of consciousness is determined by a comparison of the directional feedback connectivity to a baseline feedback connectivity.

18. The system of claim 10, further comprising:
the one or more sensors operatively coupled to the brain to monitor a feedforward activity between the second region of the brain and the first region of the brain;
the analyzing routine, which when executed on the processor, analyzes the monitored feedforward activity to determine a directional feedforward connectivity; and,
the indicating routine, which when executed on the processor, indicates the level of consciousness to a user based on a comparison of the directional feedback connectivity and the directional feedforward connectivity.

19. A computer-readable storage medium comprising computer-readable instructions stored thereon and to be executed on a processor of a system for assessing causal signaling in the brain during states of consciousness, the stored instructions comprising:
monitoring a feedback activity between a first region of the brain and a second region of the brain;
analyzing the monitored feedback activity between the first region and the second region to determine a directional feedback connectivity; and,
indicating a level of consciousness to a user.

20. The computer readable medium of claim 19, where the stored instructions further comprise indicating to a user a level of consciousness based on the determined feedback connectivity.

21. The computer readable medium of claim 19, where the stored instructions further comprise:
monitoring a feedforward activity between the second region of the brain and the first region of the brain;
analyzing the monitored feedforward activity to determine a directional feedforward connectivity, wherein the second region is a parietal region of the brain and the first region is a frontal region of the brain; and,
wherein the indicated level of consciousness is determined by a comparison between the directional feedback connectivity and the directional feedforward connectivity.
